(19) United States
(12) Patent Application Publication
(10) Pub. No.: US 2025/0367228 A1
(43) Pub. Date: Dec. 4, 2025

(54) FORMULATIONS FOR BOOSTING NAD+ USING MULTIPLE BIOLOGICAL PATHWAYS CONCURRENTLY TO OVERCOME ENZYMATIC DEGRADATION AND IMPROVE DOWNSTREAM BENEFITS

(71) Applicants: Nicholas Bitz, Irvine, CA (US); Angelica S. Vrablic, Delray Beach, FL (US); Daniel C. Obegi, El Segundo, CA (US)

(72) Inventors: Nicholas Bitz, Irvine, CA (US); Angelica S. Vrablic, Delray Beach, FL (US); Daniel C. Obegi, El Segundo, CA (US)

(73) Assignee: PROHEALTH, Carpinteria, CA (US)

(21) Appl. No.: 19/297,529

(22) Filed: Aug. 12, 2025

Related U.S. Application Data
(63) Continuation-in-part of application No. 19/005,938, filed on Dec. 30, 2024, now abandoned.
(60) Provisional application No. 63/616,286, filed on Dec. 29, 2023.

(51) Int. Cl. A61K 31/706; A61K 31/205; A61K 31/352; A61K 31/405; A61K 31/455; A61K 31/519; A61K 31/714; A61K 36/258

(52) U.S. Cl. CPC A61K 31/706; A61K 31/205; A61K 31/352; A61K 31/405; A61K 31/455; A61K 31/519; A61K 31/714; A61K 36/258

(57) ABSTRACT

Orally-ingestible supplement formulations for boosting Nicotinamide adenine dinucleotide (NAD+) in a human. The formulations provide a triple boost for NAD+ that will provide a) NAD+ boosters to increase natural production, b) a source of NAD+ and c) inhibitors to keep NAD+ from being metabolized. The formulations have one of each of: 1) an NAD precursor and/or reduced NAD precursor, 2) an NAD booster, 3) a CD38 Inhibitor, and 4) Reduced NAD precursors.

### RELATED APPLICATIONS

The present application is a continuation-in-part of U.S. Ser. No. 19/005,938, filed Dec. 30, 2024, which claims priority to prior U.S. provisional Ser. No. 63/616,286, filed Dec. 29, 2023, the contents of which are expressly incorporated herein.

### FIELD OF THE INVENTION

The present application relates generally to orally-ingestible formulations for boosting NAD+ (Nicotinamide adenine dinucleotide) in a human.

### BACKGROUND OF THE INVENTION

NAD+ (Nicotinamide adenine dinucleotide, often just “NAD”) is a coenzyme found in every mammalian cell that's critical for over 200 biological processes. NAD+ is so important that death follows instantly without NAD+. NAD+ declines as we age at a rate of about 1% per year and as it declines cell function declines which leads to aging and disease.

Despite a large number of supplements and other compositions intended to boost the presence of NAD+ in humans, there remains a need for a more efficient formulation because current formulations are rate limited, and do not account for degradation/consumption pathways which creates an upper limit for efficacy.

### SUMMARY OF THE INVENTION

The present application describes orally-ingestible supplement or drug formulations for boosting NAD+ (Nicotinamide adenine dinucleotide) in a human. The formulations provide a triple boost for NAD+ that will provide a) NAD+ boosters to increase natural production, b) an NAD+ precursor as a source of NAD+ and c) inhibitors to keep NAD+ from being metabolized.

One aspect disclosed is to make a better NAD+ booster in the form of an orally-ingestible formulation by combining at least 3 and possibly 4 different ingredients that each use a distinct metabolic pathway. For example, at least three of each of:
a NAD precursor,
a NAD booster,
a CD38 Inhibitor, and
a Reduced NAD precursor.

In the orally-ingestible formulation, the NAD precursor may be selected from the group consisting of nicotinic acid and tryptophan, the NAD booster may be selected from the group consisting of NR (Nicotinamide riboside) and NMN (Nicotinamide mononucleotide), the CD38 inhibitor may be selected from the group consisting of quercetin, apigenin, and PNGL (Panax notoginseng leaf saponins), and the reduced NAD precursor may be selected from NRH (Dihydronicotinamide riboside) and Reduced nicotinamide mononucleotide (NMNH).

In the orally-ingestible formulation, the ingredients may be provided in ranges:
a. NAD precursor: 10-60 mg,
b. NAD booster: 250-1000 mg,
c. CD38 inhibitor: 10-200 mg, and
d. Reduced NAD precursor: 100-500 mg

In the orally-ingestible formulation, the NAD precursor may be provided in the range 10-60 mg.

In the orally-ingestible formulation, the NAD booster may be provided in the range 250-1000 mg.

In the orally-ingestible formulation, the CD38 inhibitor may be provided in the range 10-200 mg.

In the orally-ingestible formulation, the Reduced NAD precursor may be provided in the range 100-500 mg.

The formulation of claim 1, wherein the ingredients may be combined in a single capsule, powder, or oral liquid dosage form. The formulation may further comprise at least one methylated B-vitamin or a methyl donor selected from the group consisting of trimethylglycine (TMG), methylcobalamin, and methylfolate, to support NAD⁺ biosynthesis via methylation pathways.

A method is disclosed for boosting NAD+ in a human in need thereof by orally self-administering a therapeutically effective amount of a formulation according to any of the ingredients above. The method may include administering the therapeutically effective amount once daily for at least 14 consecutive days. The method may include administering the therapeutically effective amount to a human over the age of 40. In the method, the formulation may comprise at least one ingredient that has been shown in a randomized, double-blind, placebo-controlled human clinical study to significantly increase plasma NAD+ levels.

### BRIEF DESCRIPTION OF THE DRAWINGS

Features and advantages of the present invention will become appreciated as the same become better understood with reference to the specification, claims, and appended drawings wherein:

FIG. 1 is a Table showing an exemplary formulation of an orally-ingestible Triple Boost composition designed to boost NAD+ in a human.

### DETAILED DESCRIPTION OF THE PREFERRED EMBODIMENTS

The present invention provides a number of preferred formulations of supplements for boosting NAD+ (Nicotinamide adenine dinucleotide) in a human.

The present application provides orally-ingestible compositions to provide a triple boost for NAD+ production that will provide a) NAD+ boosters to increase natural production, b) a source of NAD+ or NAD+ precursors that are converted in the body to NAD+ and c) inhibitors to keep NAD+ from being metabolized.

For example: Niacin is a dietary source that is metabolized to NAD+, NR (Nicotinamide riboside) is a source of NAD+, PQQ increases cellular NAD+ levels and activates sirtuins. Quercetin protects NAD+ from being metabolized and also activates sirtuins. Apigenin protects NAD+ from being metabolized. B vitamins (B2, B6 and B12) assist with methylation. PNGL are saponins from the leaves of Panax notoginseng that protect cellular NAD. Other B vitamins can assist with methylation.

Other examples: Tryptophan is a dietary source that is metabolized to NAD+, NMN (Nicotinamide mononucleotide) is a source of NAD+, and NADH (Nicotinamide Adenine Dinucleotide+H) is a source of NAD.

NAD+ can be boosted with supplements but no single supplement can boost NAD levels enough to achieve the optimal cellular function of youthful cells in adults. The NAD+ increase from supplements is limited though by the time it takes to metabolize the supplement ingredients. Our bodies can only absorb a finite amount of a nutrient per hour, and enzymatic conversion/activation is rate-limited. The formulations described herein are designed to maximize the uptake, conversion and retention of NAD+, more so than any other single pathway supplement can.

There are multiple biological pathways to increase NAD+. To help illustrate the point, a good metaphor is a freeway. On a freeway there's an upper limit to how many cars can pass each hour in each lane no matter what the qualities of the freeway and car are, but by adding lanes the volume of cars per hour increases significantly. Like how a freeway with four lanes allows four times the number of cars to pass per hour, using four biological pathways concurrently to boost NAD+ increases the total NAD+ boosting potential. By combining supplement ingredients that use multiple different pathways we're able to improve health by boosting NAD+ significantly more than what's possible by using any one pathway. Since so many diseases are caused by low NAD+ this invention has the potential to improve the lives of, and even save the lives of millions of people per year.

The four potential pathways are:
1. W—De novo pathways—Handler-Priess Pathway—NAD+ is synthesized de novo from Niacin in a three-step pathway that relies on rate-limiting enzyme Nicotinamide phosphoribosyltransferase (NAMPT); and NAD+ can be synthesized de novo from Tryptophan by the Kynurenine pathway in a 60:1 ratio.
2. X—NAD Boosters—NR—bypasses NAMPT and is intracellularly metabolized by kinases NRK1 and NRK2 to NMN. NMN (Nicotinamide mononucleotide) is transformed to NAD+ by NMNATi-3 (Nicotinamide mononucleotide adenyltransferases)
3. Y—Inhibition of NAD+ metabolizing enzymes—NAD+ase (NAD+ glycohydrolase) such as CD38 is inhibited by flavonoids quercetin and apigenin.
4. Z—Reduced precursors NRH (Dihydronicotinamide riboside) and NMNH-NRH is converted to Reduced nicotinamide mononucleotide (NMNH, also known as reduced NMN or Beta Dihydronicotinamide mononucleotide) by adenosine kinase to synthesize NAD+; NMNH is converted to NADH by NMNAT, NMNH increases cellular NADH level

Example ingredients that each use a distinct metabolic pathway are:
1. NAD precursors: Niacin Vitamin B3 as Nicotinic Acid (CAS #59-67-6), L-tryptophan
2. NAD boosters: NR (Nicotinamide Riboside) and NMN (Nicotinamide Mononucleotide)
3. CD38 Inhibitors: Quercetin and Apigenin
4. Reduced NAD precursors: NMNH and NRH

Example dose ranges of ingredients for each pathway are:
1. A: Nicotinic Acid 20 mg, Tryptophan 60 mg
2. B: NR 500 mg, NMN 500 mg
3. C: Quercetin 50 mg Apigenin 50 mg
4. D: NRH 250 mg NMNH 250 mg

Where A=(10-60 mg), B=(250-1000 mg), C=(10-200 mg), and D=(100-500 mg), where these ranges apply to either or both of the ingredients in each pathway.

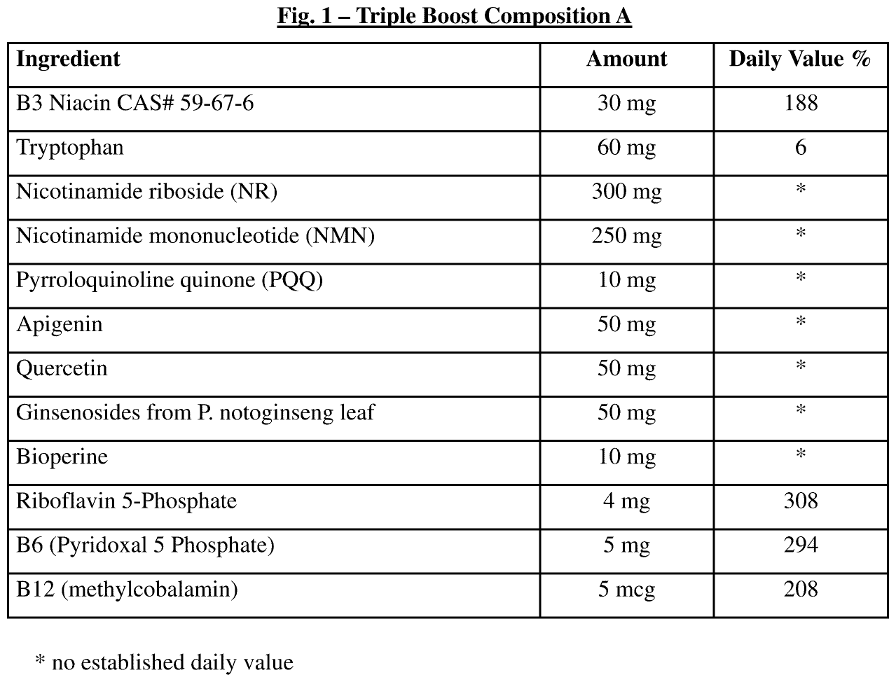

FIG. 1 is a Table showing an exemplary formulation of an orally-ingestible Triple Boost composition designed to boost NAD+ in a human, and percentage of suggested daily value for each. A formulation as in FIG. 1 may be orally ingested at a dose of 1300 mg per day (once) for a period of one month (or longer if needed), and a boost in NAD+ production and retention is found to be 50% to 100% higher than baseline amount based on human studies (observational and NAD booster interventions). For example, 1000 mg/day of NR increased NAD+ blood concentrations two-fold in elderly subjects (Elhassan Y S, et al. Cell Rep. 2019 Aug. 13; 28(7):1717-1728.e6. doi: 10.1016/j.celrep.2019.07.043. PMID: 31412242; PMCID: PMC6702140. We should expect our formula to increase NAD+ levels in the blood by at least two-fold without the need for high doses (1000 mg/day) of an NR booster. Note: no studies have been done on a triple boost formula so the increase would be the sum of the observed increases for single pathway interventions that have been tested.

As a matter of foundational support, the B vitamin niacin (B3) is part of a family of compounds that are essential for energy metabolism, cell growth and differentiation among other important functions. This “niacin family” of compounds includes (among others) nicotinamide (nicotinic acid amide) CAS #98-92-0; nicotinic acid CAS #59-67-6, NMN (Nicotinamide mononucleotide) CAS #1094-61-7; and NR (Nicotinamide riboside) CAS #2311-00-4; as well as the amino acid tryptophan which can be converted to nicotinamide. (Institute of Medicine. 2000. Dietary Reference Intakes: For Thiamin, Riboflavin, Niacin, Vitamin B₆, Folate, Vitamin B₁₂, Pantothenic Acid, Biotin and Choline. Washington, D.C.: The National Academies Press.)

Overall, the most important function is the production of nicotinamide adenine dinucleotide, NAD, a coenzyme (molecule that binds with proteins) present in all living cells. NAD acts as a hydride ion acceptor or donor in many biological redox reactions. The most well-known function of NAD is the transferring of electrons to produce ATP. (Institute of Medicine. 2000. Dietary Reference Intakes.)

NAD also activates sirtuins, enzymes associated with a wide variety of functions related to metabolism and longevity, and PARPs enzymes associated with DNA repair and cell growth/function. (Institute of Medicine. 2000. Dietary Reference Intakes.)

Production of NAD: In the cell, NAD can be produced via several different pathways. It can be synthesized de-novo from the amino acid tryptophan by the Kynurenine pathway in a 60:1 ratio. NAD can be synthesized from the B3 vitamin family substrates, nicotinic acid or niacinamide via the Priess-Handler pathway that relies on the rate-limiting enzyme Nicotinamide phosphoribosyltransferase (NAMPT) to convert niacin to NMN (Nicotinamide mononucleotide) which is then converted to NAD via NMNAT (NMN adenyltransferases). Finally, NR can be phosphorylated by the nicotinamide riboside kinase 1 and 2 (NRK1 and 2) pathway to NMN which is then converted to NAD. (Institute of Medicine. 2000. Dietary Reference Intakes.)

NAD is consumed and degraded to generate nicotinamide and ADP-ribose by several enzymes such as PARPS, and NAD hydrolases. CD38 is the main cellular NAD hydrolase in mammalian tissues and regulates cellular levels of NAD. (Escande C, Nin V, Price N L, Capellini V, Gomes A P, Barbosa M T, O'Neil L, White T A, Sinclair D A, Chini E N. Flavonoid apigenin is an inhibitor of the NAD+ase (NAD+ glycohydrolase) such as CD38: implications for cellular NAD+ metabolism, protein acetylation, and treatment of metabolic syndrome. Diabetes. 2013 April; 62(4):1084-93. doi: 10.2337/db12-1139. Epub 2012 Nov. 19. PMID: 23172919; PMCID: PMC3609577.)

CD38 is inhibited by flavonoids such as quercetin and apigenin. (Escande, C. et al. Diabetes. 2013)

Boosting NAD levels: B3 analogues such as niacinamide and nicotinic acid are first-order NAD boosters that increase natural production, while NR and NMN are likely more efficient because they can bypass the rate-limiting enzyme NAMPT (Nicotinamide phosphoribosyltransferase) of the Handler-Priess pathway. However, NAD can also be consumed by NAD hydrolases such as CD38, so inhibition of the CD38 enzyme will sustain the increase and availability of NAD by decreasing consumption and degradation.

Suggested Boosters: A triple boost for NAD+ production will provide NAD+ boosters to increase natural production, a source of NAD+ that bypasses the rate-limiting NAMPT enzyme in the Priess-Handler pathway and inhibitors of NAD hydrolases such as CD38 to keep NAD+ from being metabolized.

Niacin is a dietary source that is metabolized to NAD+, as is the amino acid tryptophan. NR is a source of NAD+, and flavonoids such as quercetin and apigenin protect NAD+ from being metabolized by NADases such as CD38. (Escande, C. et al. Diabetes. 2013)

Quercetin also activates sirtuins. Additionally, B vitamins (B2, B6 and B12) provide coenzyme support for nucleotide metabolism and methylation. (Institute of Medicine. 2000. Dietary Reference Intakes.)

PNGL are saponins from the leaves of Panax notoginseng that protect cellular NAD. (Zhou N, Tang Y, Keep R F, Ma X, Xiang J. Antioxidative effects of Panax notoginseng saponins in brain cells. Phytomedicine. 2014 Sep. 15; 21(10):1189-95. doi: 10.1016/j.phymed.2014.05.004. Epub 2014 June PMID: 24916704; PMCID: PMC4111966.)

While the invention has been described in its preferred embodiments, it is to be understood that the words that have been used are words of description and not of limitation. Therefore, changes may be made within the appended claims without departing from the true scope of the invention.

What is claimed is:

1. An orally-ingestible formulation for boosting NAD+ in a human by combining 3 or more different ingredients that use at least 3 distinct metabolic pathways selected from the group consisting of the following:
a. a NAD precursor,
b. a NAD booster,
c. a CD38 Inhibitor, and
d. a Reduced NAD precursor.

2. The formulation of claim 1, wherein the NAD precursor is selected from the group consisting of nicotinic acid and tryptophan.

3. The formulation of claim 1, wherein the NAD booster is selected from the group consisting of NR (Nicotinamide riboside) and NMN (Nicotinamide mononucleotide).

4. The formulation of claim 1, wherein the CD38 inhibitor is selected from the group consisting of quercetin, apigenin, and PNGL (Panax notoginseng leaf saponins).

5. The formulation of claim 1, wherein the reduced NAD precursor is selected from NRH (Dihydronicotinamide riboside) and Reduced nicotinamide mononucleotide (NMNH).

6. The formulation of claim 1, wherein the ingredients are provided in ranges:
a. NAD precursor: 10-60 mg,
b. NAD booster: 250-1000 mg,
c. CD38 inhibitor: 10-200 mg, and
d. Reduced NAD precursor: 100-500 mg

7. The formulation of claim 1, wherein the NAD precursor is provided in the range 10-60 mg.

8. The formulation of claim 7, wherein the NAD booster is provided in the range 250-1000 mg.

9. The formulation of claim 7, wherein the CD38 inhibitor is provided in the range 10-200 mg.

10. The formulation of claim 7, wherein the Reduced NAD precursor is provided in the range 100-500 mg.

11. The formulation of claim 7, wherein the NAD booster is provided in the range 250-1000 mg.

12. The formulation of claim 1, wherein the CD38 inhibitor is provided in the range 10-200 mg.

13. The formulation of claim 1, wherein the Reduced NAD precursor is provided in the range 100-500 mg.

14. The formulation of claim 1, wherein the ingredients are combined in a single capsule, powder, or oral liquid dosage form.

15. The formulation of claim 1, further comprising at least one methylated B-vitamin or a methyl donor selected from the group consisting of trimethylglycine (TMG), methylcobalamin, and methylfolate, to support NAD⁺ biosynthesis via methylation pathways.

16. A method for boosting NAD+ in a human in need thereof by orally self-administering a therapeutically effective amount of a formulation according to claim 1.

17. The method of claim 16, including administering the therapeutically effective amount once daily for at least 14 consecutive days.

18. The method of claim 16, including administering the therapeutically effective amount to a human over the age of 40.

19. The method of claim 16, wherein the formulation comprises at least one ingredient that has been shown in a randomized, double-blind, placebo-controlled human clinical study to significantly increase plasma NAD+ levels.
